(19) United States
(12) Patent Application Publication
(10) Pub. No.: US 2015/0342908 A1
(43) Pub. Date: Dec. 3, 2015

(54) SECOAPORPHINE ALKALOID DERIVATIVE FOR ACTIVATING AMP-DEPENDENT PROTEIN KINASE

(71) Applicant: ZIH YUAN TANG Biotechnology Co., Ltd, Taipei City (TW)

(72) Inventors: Shoei-Sheng Lee, Taipei City (TW); Ming-Jai Su, Taipei City (TW); Chi-Huan Yeh, Miaoli County (TW); Cheng-Yen Tsai, Taichung City (TW); Chi-Tun Ruan, New Taipei City (TW); Chao-Min Hsu, New Taipei City (TW)

(21) Appl. No.: 14/492,652

(22) Filed: Sep. 22, 2014

(30) Foreign Application Priority Data
May 30, 2014 (TW) 103119119

(51) Int. Cl. A61K 31/137

(52) U.S. Cl. CPC A61K 31/137

(57) ABSTRACT

The present invention provides a method for activating the AMP-dependent protein kinase (AMPK) in a subject comprising administering the subject with a pharmaceutical composition comprising a pharmaceutically acceptable carrier and a therapeutically effective amount of a compound having the general Formula I, preferably 1,10-dihydroxyaporphine. The pharmaceutical composition is able to activate AMPK, and thus is effective in the regulation of cell growth and metabolism, and the treatment of AMPK associated diseases.

### FIELD OF THE INVENTION

The present invention provides a method for activating AMP-dependent protein kinase (AMPK) using a secoaporphine alkaloid derivative.

### BACKGROUND OF THE INVENTION

Secoaporphine alkaloid generally exists in plants such as Papaveraceae, and Sapindaceae, which structure belongs to aminoethylphenanthrene, and the aminoethylphenanthrene can be obtained by synthesis of isoquinoline from tyrosine in vivo. The pharmacological activity of secoaporphine alkaloid includes analgesia, cough reliever, or emetics. Secoaporphine alkaloid also be demonstrated the effect in treatment of cardiovascular diseases (Su et al., U.S. Pat. No. 5,594,033)

AMP-dependent protein kinase (AMPK) is a metabolic or energy sensor of the cells, characterized in that it can bind with AMP and maintain the balance between the generation and consumption of ATP through AMP, and thus maintain the balance of energy metabolism. Meanwhile, AMPK can also modulate cell growth and proliferation, establish and stabilize cell polarity, regulate animal lifespan, and modulate physiological rhythms. In recent years, targeting AMPK activation has become one of the key points in pharmaceutical development. Therefore, the pharmaceutical industry is actively pursuing the development of new AMPK activators.

### BRIEF SUMMARY OF THE INVENTION

It is unexpectedly found in the present invention that certain secoaporphine alkaloid derivative, such as N-allylsecoboldine, is effective in the activation of AMPK.

In one aspect, the present invention provides a method for activating the AMP-dependent protein kinase (AMPK) in a subject comprising administering the subject with a pharmaceutical composition comprising a pharmaceutically acceptable carrier and a therapeutically effective amount of a compound having the general Formula I or a pharmaceutically acceptable salt:

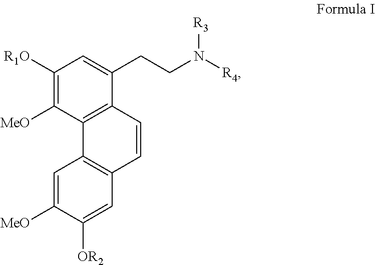

wherein R₁, R₂ and R₃ are each independently H or alkyl group; R₄ is H, alkyl, allyl (—CH₂CHCH₂) or benzyl group.

In one example of the present invention, said compound having Formula I is N-allylsecoboldine or pharmaceutically acceptable salt thereof.

In another example of the present invention, said compound having Formula I is N-allylsecoboldine.

In still another aspect, the present invention provides a method for treating the AMPK-related disease in a subject comprising administering the subject with a pharmaceutical composition comprising a pharmaceutically acceptable carrier and a therapeutically effective amount of a compound having the general Formula I or a pharmaceutically acceptable salt of the present invention, wherein the treatment is achieved through the activation of AMPK by the compound having the general Formula I or a pharmaceutically acceptable salt; wherein said AMPK-related disease is cancer. The compound having Formula I or a pharmaceutically acceptable salt of the present invention also has an effect in anti-inflammation or promoting wound healing.

Those and other aspects of the present invention may be further clarified by the following descriptions and drawings of preferred embodiments. Although there may be changes or modifications therein, they would not betray the spirit and scope of the novel ideas disclosed in the present invention.

### BRIEF DESCRIPTION OF THE DRAWINGS

The drawings presenting the preferred embodiments of the present invention are aimed at explaining the present invention. It should be understood that the present invention is not limited to the preferred embodiments shown. The data in the figures and examples are shown as mean±standard deviation (SD), determined by the paired t-test. Significant differences are shown as follows: *: P<0.05; **: P<0.01; #: P=0.067.

FIG. 1 shows the influence of N-allylsecoboldine on AMPK phosphorylation, wherein AMPK activity tests were conducted in C₂C₁₂ cells with various concentrations (1, 3, and 10 μM) of N-allylsecoboldine.

### DETAILED DESCRIPTION OF THE INVENTION

Unless defined otherwise, all technical and scientific terms used herein have the same meanings as commonly understood by one of skill in the art to which this invention belongs.

Unless clearly specified herein, meanings of the articles “a,” “an,” and “said” all include the plural form of “more than one.” Therefore, for example, when the term “a component” is used, it includes multiple said components and equivalents known to those of common knowledge in said field.

As used herein, the term “activating” or “activation” refers to an action to make a molecule active, or to cause a molecule to function or act. In the invention, the activation means that the compound cause AMPK to function in the subject.

As used herein, the term “subject” refers to a human or a mammal, such as a patient, a companion animal (e.g., dog, cat, and the like), a farm animal (e.g., cow, sheep, pig, horse, and the like) or a laboratory animal (e.g., rat, mouse, rabbit, and the like).

The term “AMPK” as used herein is the abbreviation of AMP-dependent protein kinase, which refers to a type of protein kinase that regulates energy metabolism in cells, being the major regulatory factor in many biological processes. The signaling pathway of AMPK includes metabolism of glucose and lipids, and influences the expression of relevant genes and proteins. When AMPK is phosphorylated, its activity will be stimulated and downstream proteins in the AMPK signaling pathway will be further regulated, and thereby metabolism in the liver, skeletal muscles, heart, lipid tissues and pancreas will be regulated. Therefore, medications effective in AMPK activation can be potentially effective in treating many diseases, such as metabolism diseases (such as diabetes), cancer, and cardiovascular diseases (such as atherosclerosis and ischemic heart disease). They can also be used for anti-inflammation or promoting wound healing. The mode of action of AMPK activators including anti-inflammatory activities in vascular endothelial cells has been established in some preclinical and clinical findings; therefore, AMPK is also considered as a drug target in treating cardiovascular and metabolic diseases.

The term “alkyl group” used herein refers to linear or branched monovalent hydrocarbons containing 1-20 carbon atoms, such as alkyl groups with 1-10 carbons, preferably alkyl groups with 1-6 carbons, more preferably alkyl groups with 1-3 carbons. Examples of alkyl groups include, but not limited to, methyl, ethyl, n-propyl, isopropyl, n-butyl, isobutyl, and t-butyl.

As evidenced in the examples, N-allylsecoboldine has an excellent effect in AMPK activation. Accordingly, the present invention provides a method for activating the AMP-dependent protein kinase (AMPK).

According to the invention, the active compound has the general Formula I:

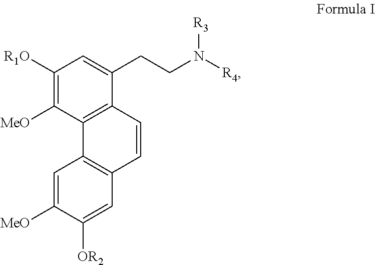

wherein R₁, R₂ and R₃ are each independently H or alkyl group; R₄ is H, alkyl, allyl (—CH₂CHCH₂) or benzyl group.

An embodiment of the active compound of the present invention is the compound having the general Formula I, wherein R₁═R₂═H, R₃═CH₃ (methyl), and R₄═CH₂CHCH₂ (allyl), which compound is N-allylsecoboldine having the following formula:

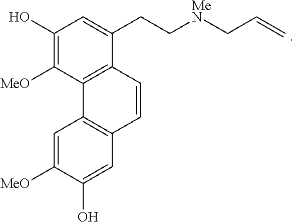

As shown in the examples of the present invention, the compound having Formula I of the present invention, such as N-allylsecoboldine, has an effect of activating AMPK.

In addition, the compounds having Formula I of the present invention are effective in AMPK activation so as to achieve the effect of treatment, and thus are useful in treating AMPK-related diseases. For examples, AMPK-related disease is selected from the group consisting of cancer, cardiovascular diseases, and metabolism diseases. In addition, it is also considered to have an effect in anti-inflammation or promoting wound healing.

According to the present invention, said compound having Formula I can be formulated into any forms of medications that are well known or commonly used in the pharmaceutical field, and can be prepared into a composition, according to any techniques well known in the pharmaceutical field, comprising a therapeutically effective amount of said compound in combination with a commonly used carrier or a pharmaceutically acceptable carrier.

The term “carrier” or “pharmaceutically acceptable carrier” used herein includes, but not limited to, pharmaceutically acceptable excipients, fillers, diluents, or the like, including those well known to one of ordinary skills in the pharmaceutical field.

The present invention is explained in the above description of the invention and the following examples, which should not be used to restrict the scope of the present invention.

### Example 1

### Preparation of N-allylsecoboldine

The solution of boldine (1.0 g), N,N-dimethylformamide (DMF) (30 mL), and allyl bromide (1.2 mL) was heated at 100-110° C. for 2 hours. The resultant product was purified to obtain N-allylsecoboldine. In particular, the N-allylsecoboldine can be prepared as mentioned in Taiwan Patent No. 1105255, the entire content of which are incorporated by reference herein.

### Example 2

### Evaluation of the Effect of N-allylsecoboldine in AMPK Activation

C₂C₁₂ skeletal myoblast cell line was purchased from the Food Industry Research and Development Institute (Hsinchu, Taiwan). The C₂C₁₂ cell line was a cell line obtained from culturing the leg skeletal muscle of adult C3H mice in a cell incubator under 95% O₂, 5% CO₂, and 37° C. The cells were cultured in a DMEM medium (Gibco/Invitrogen, Carlsbad, Calif.) containing 4.5 mg/mL glucose, 10% fetal bovine serum (FBS; Gibco/Invitrogen, Carlsbad, Calif.), and an antibiotic solution (with final concentrations of penicillin=100 IU/mL and streptomycin=100 μg/mL). When the C₂C₁₂ myoblast cells have proliferated to cover seven-tenths of the area in the petri dish, the fetal bovine serum was replaced by 2% horse serum (Gibco/Invitrogen, Carlsbad, Calif.) so as to induce the C₂C₁₂ myoblast cells to become the multi-nuclei myocytes. The C₂C₁₂ myoblast cells differentiated into myocytes in 4 days, and the culture medium of the cells was replaced with no-serum DMEM 24 hours before the experiments so as to reduce metabolism of the cells.

The C₂C₁₂ myocytes were treated respectively with 1 μM, 3 μM and 10 μM N-allylsecoboldine for 5 and 15 minutes. Then the C₂C₁₂ myocytes were rinsed with PBS buffer solution, and RIPA buffer solution containing protease inhibitors (20 mM Tris-HCl (pH 7.4), 100 mM NaCl, 1 mM EDTA, 1 mM EGTA, 0.1% SDS, 0.5% sodium deoxycholate, 1% NP-40, and 100× protease inhibitor cocktail) was added into the cells. The cells were collected and centrifuged on ice. Then, the concentrations of the supernatant samples were adjusted to be the same.

The supernatant samples were put through vertical electrophoresis isolation with 8% SDS-PAGE, and the isolated proteins were transferred onto a PVDF blotting membrane. After blotting was completed, the PVDF blotting membrane was removed and blocked for 1 hour under room temperature with a blocking buffer of TBST (Tris-buffered saline with Tween-20) with 5% non-fat milk. Then, the PVDF blotting membrane was placed into a 5% BSA and TBST solution containing the primary monoclonal antibodies Phospho-AMPKα (Thr172) (Cell Signaling) (1:1000) and AMPKα (Thr172) (Cell Signaling) (1:1000), respectively, and the primary immunoblotting reaction was performed under 4° C. Then, the PVDF blotting membrane was rinsed 3 times with TBST, and a TBST solution containing the secondary antibody goat anti-rabbit IgG (Perkin Elmer) (1:10000) was added to perform the secondary reaction for one hour under room temperature. Finally, the membrane was rinsed 3 times with TBST before ECL (enhanced chemiluminescence) was added to present color.

Results

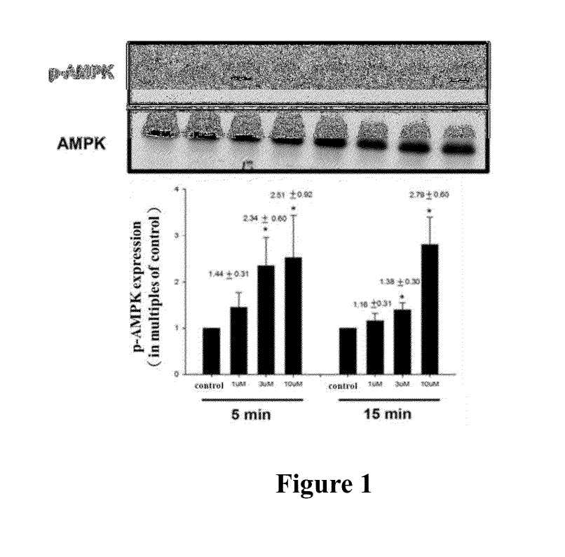

As shown in FIG. 1, after the C₂C₁₂ cells were treated with 3 μM and 10 μM N-allylsecoboldine for 5 minutes, the AMPK phosphorylation in these cells increased to 2.34 and 2.51 times of that in the control cells, respectively. After the C₂C₁₂ cells were treated for 15 minutes, the AMPK phosphorylation in the cells increased to 1.38 and 2.79 times of that in the control cells. These results shown that N-allylsecoboldine has an excellent effect in AMPK activation.

Based on the results in the examples, one can see that secoaporphine alkaloid has an excellent effect in AMPK activation. In particularly, the N-allylsecoboldine has an excellent effect in AMPK activation, wherein the 3 μM N-allylsecoboldine for 5 min treatment has a pretty good effect in AMPK activation, and 10 μM N-allylsecoboldine for 15 min treatment can provide the best effect in AMPK activation.

We claim:

1. A method for activating the AMP-dependent protein kinase (AMPK) in a subject comprising administering the subject with a pharmaceutical composition comprising a pharmaceutically acceptable carrier and a therapeutically effective amount of a compound having the general Formula I or a pharmaceutically acceptable salt: 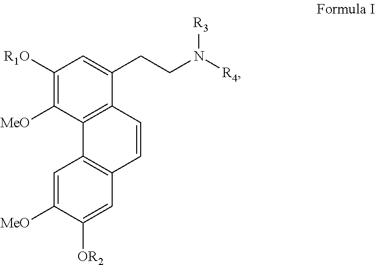
wherein R₁, R₂ and R₃ are each independently H or alkyl group; R₄ is H, alkyl, allyl (—CH₂CHCH₂) or benzyl group.

2. The method of claim 1, wherein the compound having Formula I is N-allylsecoboldine or pharmaceutically acceptable salt thereof.

3. The method of claim 2, wherein the compound having Formula I is N-allylsecoboldine.

4. A method for treating the AMPK-related disease in a subject comprising administering the subject with a pharmaceutical composition comprising a pharmaceutically acceptable carrier and a therapeutically effective amount of a compound having the general Formula I or a pharmaceutically acceptable salt as set forth in claim 1.

5. The method of claim 4, wherein the compound having Formula I is N-allylsecoboldine or pharmaceutically acceptable salt thereof.

6. The method of claim 5, wherein the compound having Formula I is N-allylsecoboldine.

7. The method of claim 4, wherein said AMPK-related disease is cancer.

8. The method of claim 4, wherein said pharmaceutical composition has an effect in anti-inflammation or promoting wound healing.
